(19) United States
(12) Patent Application Publication
(10) Pub. No.: US 2025/0114558 A1
(43) Pub. Date: Apr. 10, 2025

(54) Systems and Methods for Detection and Remediation of Triggers

(71) Applicant: Comcast Cable Communications, LLC, Philadelphia, PA (US)

(72) Inventor: Mohamed Zakaria Sahul, Voorhees, NJ (US)

(21) Appl. No.: 18/482,602

(22) Filed: Oct. 6, 2023

(51) Int. Cl. A61M 21/02; A61M 21/00; G06F 3/16; G06V 20/40; G10K 11/178

(52) U.S. Cl. CPC A61M 21/02; G06F 3/165; G06V 20/44; G10K 11/1783; A61M 2021/0027; A61M 2021/005; G10K 2210/1081; G10K 2210/3016

(57) ABSTRACT

Systems, apparatuses, and methods are described for detection and remediation of triggers from a combination of a user's environment and content being output to the user. Based on the detected triggers and/or user agitation, remedial actions may be deployed to prevent or alleviate user agitation, which may include adjusting environmental devices and/or modifying content being output to the user.

### BACKGROUND

Content may comprise conditions and/or events that trigger individuals with certain disabilities, conditions, and/or sensitivities (e.g., autism). Triggering content may be detected in content via analysis of content video, audio, and/or metadata. Viewing triggering content may trigger emotional and/or psychological responses in some individuals. Triggering content may include content comprising loud noises, bright lights, violence, strong language, flashing lights, and/or other events.

### SUMMARY

The following summary presents a simplified summary of certain features.

The summary is not an extensive overview and is not intended to identify key or critical elements. Systems, apparatuses, and methods are described for detection and remediation of detected triggers to help prevent and/or minimize harmful reactions to triggering events. Triggers may be detected via various sensors (e.g., cameras, microphones, motion sensors, wearable devices, etc.). Sensors may detect indications of triggering characteristics in ambient conditions and in content, which may increase user agitation (e.g., indicated by body movements and/or vocal utterances or phrases). Based on comparing the detected triggers in the content and/or environment and/or user agitation, remedial actions may be deployed to prevent or alleviate agitation. Remedial actions may include adjusting environmental devices and/or modifying content being output to the user. For example, if a movie viewer has ASD and is triggered by scenes of violence, then as a violent scene approaches in the movie, various remedial actions may be taken such as adjusting ambient lighting in the room, playing soothing background audio, and replacing portions of the violent scene with alternative content. The remedial actions may also be based on observed physical signs of stress. If the viewer is already showing signs that he/she is in an agitated state, then different remedial actions may be taken, such as adjusting the lighting earlier, activating a massage chair, etc.

These and other features and advantages are described in greater detail below.

### BRIEF DESCRIPTION OF THE DRAWINGS

Some features are shown by way of example, and not by limitation, in the accompanying drawings. In the drawings, like numerals reference similar elements.

FIG. 1 shows an example communication network.

FIG. 2 shows hardware elements of a computing device.

FIG. 3 shows an example environment for detecting and remediating triggers.

FIG. 4 is an example table showing detected triggers and remedial actions.

FIGS. 5A-5D are flow charts showing an example method for remediation based on detected triggers.

### DETAILED DESCRIPTION

The accompanying drawings, which form a part hereof, show examples of the disclosure. It is to be understood that the examples shown in the drawings and/or discussed herein are non-exclusive and that there are other examples of how the disclosure may be practiced.

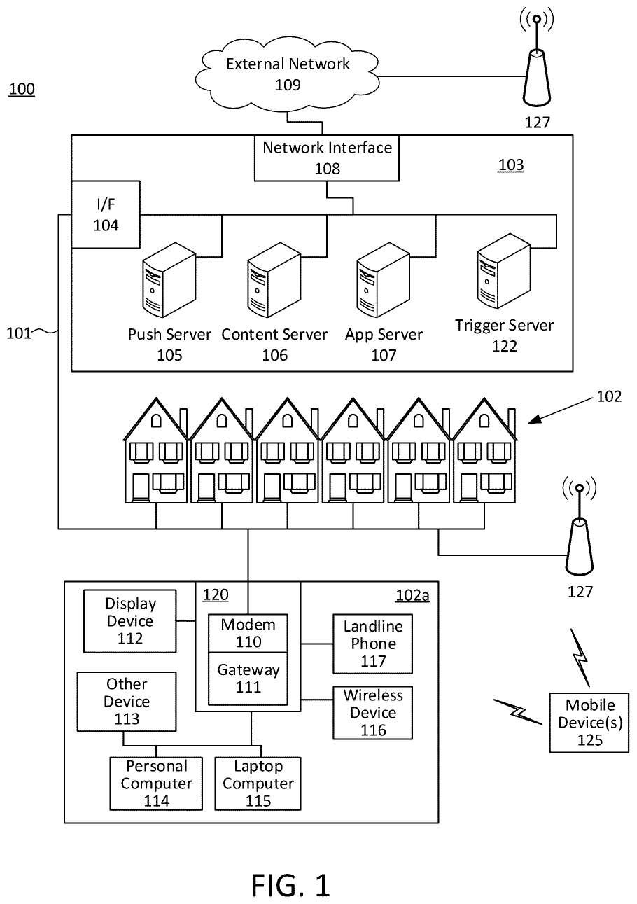

FIG. 1 shows an example communication network 100 in which features described herein may be implemented. The communication network 100 may comprise one or more information distribution networks of any type, such as, without limitation, a telephone network, a wireless network (e.g., an LTE network, a 5G network, a WiFi IEEE 802.11 network, a WiMAX network, a satellite network, and/or any other network for wireless communication), an optical fiber network, a coaxial cable network, and/or a hybrid fiber/coax distribution network. The communication network 100 may use a series of interconnected communication links 101 (e.g., coaxial cables, optical fibers, wireless links, etc.) to connect multiple premises 102 (e.g., businesses, homes, consumer dwellings, train stations, airports, etc.) to a local office 103 (e.g., a headend). The local office 103 may send downstream information signals and receive upstream information signals via the communication links 101. Each of the premises 102 may comprise devices, described below, to receive, send, and/or otherwise process those signals and information contained therein.

The communication links 101 may originate from the local office 103 and may comprise components not shown, such as splitters, filters, amplifiers, etc., to help convey signals clearly. The communication links 101 may be coupled to one or more wireless access points 127 configured to communicate with one or more mobile devices 125 via one or more wireless networks. The mobile devices 125 may comprise smart phones, tablets or laptop computers with wireless transceivers, tablets or laptop computers communicatively coupled to other devices with wireless transceivers, and/or any other type of device configured to communicate via a wireless network.

The local office 103 may comprise an interface 104. The interface 104 may comprise one or more computing devices configured to send information downstream to, and to receive information upstream from, devices communicating with the local office 103 via the communications links 101. The interface 104 may be configured to manage communications among those devices, to manage communications between those devices and backend devices such as servers 105-107 and 122, and/or to manage communications between those devices and one or more external networks 109. The interface 104 may, for example, comprise one or more routers, one or more base stations, one or more optical line terminals (OLTs), one or more termination systems (e.g., a modular cable modem termination system (M-CMTS) or an integrated cable modem termination system (I-CMTS)), one or more digital subscriber line access modules (DSLAMs), and/or any other computing device(s). The local office 103 may comprise one or more network interfaces 108 that comprise circuitry needed to communicate via the external networks 109. The external networks 109 may comprise networks of Internet devices, telephone networks, wireless networks, wired networks, fiber optic networks, and/or any other desired network. The local office 103 may also or alternatively communicate with the mobile devices 125 via the interface 108 and one or more of the external networks 109, e.g., via one or more of the wireless access points 127.

The push notification server 105 may be configured to generate push notifications to deliver information to devices in the premises 102 and/or to the mobile devices 125. The content server 106 may be configured to provide content to devices in the premises 102 and/or to the mobile devices 125. This content may comprise, for example, video, audio, text, web pages, images, files, etc. The content server 106 (or, alternatively, an authentication server) may comprise software to validate user identities and entitlements, to locate and retrieve requested content, and/or to initiate delivery (e.g., streaming) of the content. The application server 107 may be configured to offer any desired service. For example, an application server may be responsible for collecting, and generating a download of, information for electronic program guide listings. Another application server may be responsible for monitoring user viewing habits and collecting information from that monitoring for use in selecting advertisements. Yet another application server may be responsible for formatting and inserting advertisements in a video stream being transmitted to devices in the premises 102 and/or to the mobile devices 125. The local office 103 may comprise additional servers, such as the trigger server 122 (described below), additional push, content, and/or application servers, and/or other types of servers.

The trigger server 122 may comprise lists of triggers and/or types of triggers, lists of user behaviors (e.g., indicating various levels of agitation), trigger thresholds for users (e.g., predetermined levels at which remedial actions should be taken), remedial actions (e.g., environmental actions, content modification instructions, etc.), user profiles indicating triggers and/or behavioral patterns, and/or other data. Although shown separately, the push server 105, the content server 106, the application server 107, the trigger server 122, and/or other server(s) may be combined. The servers 105, 106, 107, and 122, and/or other servers, may be computing devices and may comprise memory storing data and also storing computer executable instructions that, when executed by one or more processors, cause the server(s) to perform steps described herein.

An example premises 102a may comprise an interface 120. The interface 120 may comprise circuitry used to communicate via the communication links 101. The interface 120 may comprise a modem 110, which may comprise transmitters and receivers used to communicate via the communication links 101 with the local office 103. The modem 110 may comprise, for example, a coaxial cable modem (for coaxial cable lines of the communication links 101), a fiber interface node (for fiber optic lines of the communication links 101), twisted-pair telephone modem, a wireless transceiver, and/or any other desired modem device. One modem is shown in FIG. 1, but a plurality of modems operating in parallel may be implemented within the interface 120. The interface 120 may comprise a gateway 111. The modem 110 may be connected to, or be a part of, the gateway 111. The gateway 111 may be a computing device that communicates with the modem(s) 110 to allow one or more other devices in the premises 102a to communicate with the local office 103 and/or with other devices beyond the local office 103 (e.g., via the local office 103 and the external network(s) 109). The gateway 111 may comprise a set-top box (STB), digital video recorder (DVR), a digital transport adapter (DTA), a computer server, and/or any other desired computing device.

The gateway 111 may also comprise one or more local network interfaces to communicate, via one or more local networks, with devices in the premises 102a. Such devices may comprise, e.g., display devices 112 (e.g., televisions), other devices 113 (e.g., a DVR or STB), personal computers 114, laptop computers 115, wireless devices 116 (e.g., wireless routers, wireless laptops, notebooks, tablets and netbooks, cordless phones (e.g., Digital Enhanced Cordless Telephone-DECT phones), mobile phones, mobile televisions, personal digital assistants (PDA)), landline phones 117 (e.g., Voice over Internet Protocol-VoIP phones), and any other desired devices. Example types of local networks comprise Multimedia Over Coax Alliance (MoCA) networks, Ethernet networks, networks communicating via Universal Serial Bus (USB) interfaces, wireless networks (e.g., IEEE 802.11, IEEE 802.15, Bluetooth), networks communicating via in-premises power lines, and others. The lines connecting the interface 120 with the other devices in the premises 102a may represent wired or wireless connections, as may be appropriate for the type of local network used. One or more of the devices at the premises 102a may be configured to provide wireless communications channels (e.g., IEEE 802.11 channels) to communicate with one or more of the mobile devices 125, which may be on- or off-premises.

The mobile devices 125, one or more of the devices in the premises 102a, and/or other devices may receive, store, output, and/or otherwise use assets. An asset may comprise a video, a game, one or more images, software, audio, text, webpage(s), and/or other content.

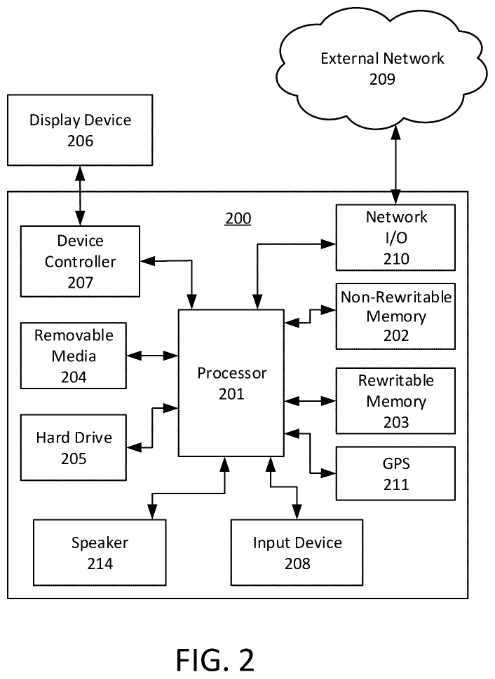

FIG. 2 shows hardware elements of a computing device 200 that may be used to implement any of the computing devices shown in FIG. 1 (e.g., the mobile devices 125, any of the devices shown in the premises 102a, any of the devices shown in the local office 103, any of the wireless access points 127, any devices with the external network 109) and any other computing devices discussed herein. The computing device 200 may comprise one or more processors 201, which may execute instructions of a computer program to perform any of the functions described herein. The instructions may be stored in a non-rewritable memory 202 such as a read-only memory (ROM), a rewritable memory 203 such as random access memory (RAM) and/or flash memory, removable media 204 (e.g., a USB drive, a compact disk (CD), a digital versatile disk (DVD)), and/or in any other type of computer-readable storage medium or memory. Instructions may also be stored in an attached (or internal) hard drive 205 or other types of storage media. The computing device 200 may comprise one or more output devices, such as a display device 206 (e.g., an external television and/or other external or internal display device) and a speaker 214, and may comprise one or more output device controllers 207, such as a video processor or a controller for an infra-red or BLUETOOTH transceiver. One or more user input devices 208 may comprise a remote control, a keyboard, a mouse, a touch screen (which may be integrated with the display device 206), microphone, etc. The computing device 200 may also comprise one or more network interfaces, such as a network input/output (I/O) interface 210 (e.g., a network card) to communicate with an external network 209. The network I/O interface 210 may be a wired interface (e.g., electrical, RF (via coax), optical (via fiber)), a wireless interface, or a combination of the two. The network I/O interface 210 may comprise a modem configured to communicate via the external network 209. The external network 209 may comprise the communication links 101 discussed above, the external network 109, an in-home network, a network provider's wireless, coaxial, fiber, or hybrid fiber/coaxial distribution system (e.g., a DOCSIS network), or any other desired network. The computing device 200 may comprise a location-detecting device, such as a global positioning system (GPS) microprocessor 211, which may be configured to receive and process global positioning signals and determine, with possible assistance from an external server and antenna, a geographic position of the computing device 200.

Although FIG. 2 shows an example hardware configuration, one or more of the elements of the computing device 200 may be implemented as software or a combination of hardware and software. Modifications may be made to add, remove, combine, divide, etc. components of the computing device 200. Additionally, the elements shown in FIG. 2 may be implemented using basic computing devices and components that have been configured to perform operations such as are described herein. For example, a memory of the computing device 200 may store computer-executable instructions that, when executed by the processor 201 and/or one or more other processors of the computing device 200, cause the computing device 200 to perform one, some, or all of the operations described herein. Such memory and processor(s) may also or alternatively be implemented through one or more Integrated Circuits (ICs). An IC may be, for example, a microprocessor that accesses programming instructions or other data stored in a ROM and/or hardwired into the IC. For example, an IC may comprise an Application Specific Integrated Circuit (ASIC) having gates and/or other logic dedicated to the calculations and other operations described herein. An IC may perform some operations based on execution of programming instructions read from ROM or RAM, with other operations hardwired into gates or other logic. Further, an IC may be configured to output image data to a display buffer.

Autism is a developmental condition caused by differences in the brain. Approximately 1 in 44 children have been identified to have autism spectrum disorder (ASD), which is associated with challenges in social communication, interaction, restricted and/or repetitive behaviors, among other challenges. The rate of occurrence of ASD is approximately 240% higher than baseline estimates established in 2000. Individuals with ASD may experience periods of increased agitation (e.g., meltdowns), which may be triggered by factors such as lights, sounds, silence, mood swings, and/or other factors.

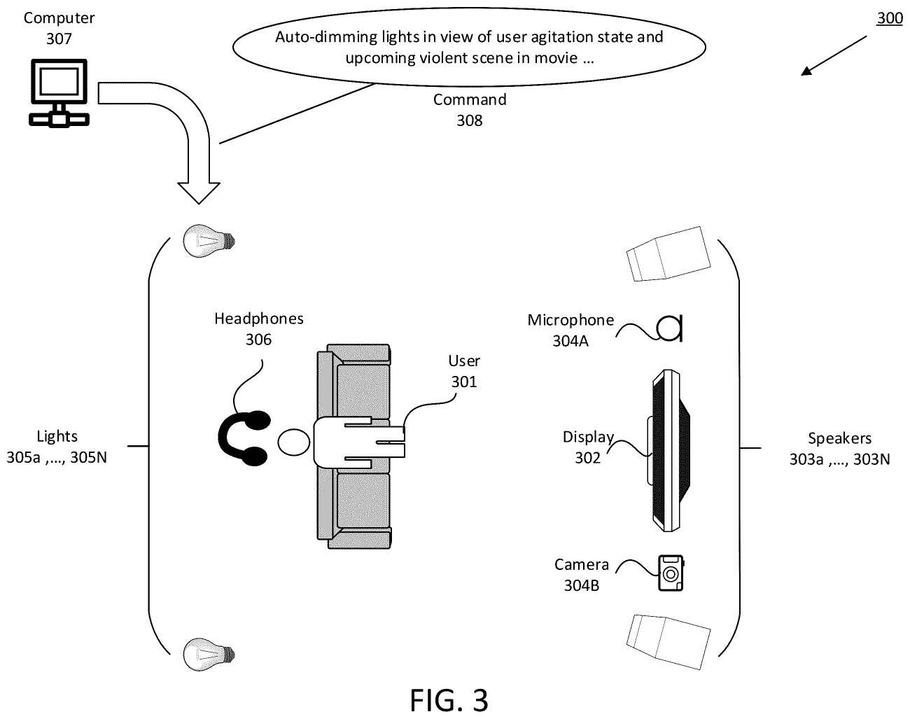

FIG. 3 shows an environment 300 for detecting and remediating triggers. The environment 300 may comprise one or more users 301, a display 302, speakers 303a . . . 303N, a microphone 304A, a camera 304B, lights 305a . . . 305N, headphones 306, a computer 307, and a command 308. In the environment 300, trigger conditions may be detected in content being output to a user, in the user's environment, and/or some combination thereof via content audio analysis, content image analysis, analysis of data from environmental sensors, and/or other methods using the microphone 304A, the camera 304B, the lights 305a . . . 305N, the computer 307, and/or other data sources. Although FIG. 3 shows one user, there may be any number of users present in the environment for whom triggers are monitored and/or remediated.

In order to detect trigger conditions, analysis of ambient environmental audio, content audio, environmental video, content video, and/or other data may be performed. Audio triggers in the environment may be detected (e.g., via sensors such as the microphone 304A). Audio triggers in content being output to a user may be detected (e.g., based on analysis of the content audio, metadata associated with the content, etc.). The levels of the audio triggers (e.g., volume level, intensity, etc.) may be determined, for example, by the computer 307. The detected audio triggers and/or the corresponding audio trigger levels may be compared to predetermined thresholds, for example, to determine whether the detected triggers meet the predetermined thresholds. The predetermined thresholds may indicate, for example, maximum trigger levels that a user can tolerate. For example, a user sensitive to loud noises may be able to tolerate sounds up to 40 dB, so their audio threshold may be set to 40 dB. If environment audio, content audio, or some combination thereof exceeds 40 dB, remedial actions may be deployed in order to prevent or alleviate agitation of the user, for example, such as modifying content audio, activating noise cancelling devices, and/or other actions. To determine whether the audio threshold has been met, environment audio and content audio may be compared.

Visual trigger conditions may be similarly detected. Visual triggers in content being output to a user may be detected (e.g., based on analysis of the content video stream, metadata associated with the content, etc.). The levels of the visual triggers (e.g., brightness level, intensity, flashing, etc.) may be determined, for example, by the computer 307. The detected visual triggers and/or the corresponding visual trigger levels may be compared to predetermined thresholds, for example, to determine whether the detected triggers meet the predetermined thresholds. The predetermined thresholds may indicate, for example, maximum trigger levels that a user can tolerate.

Users may be triggered, for example, by differences between ambient environmental conditions and content being output to the users. For example, a user may be sensitive to differences in lighting. Comparison of content and ambient conditions, for example, to determine differences in lighting may comprise comparing the brightness of content with the brightness of the ambient lighting. A triggering lighting difference may be determined, for example, if the difference between the ambient lighting and the content lighting satisfies a predetermined threshold. For example, a display outputting the content may be at 500 lumens and the ambient lighting may be low or off. The difference may be determined, for example, by the computer 307. A triggering lighting condition may be identified and/or remedial actions may be deployed.

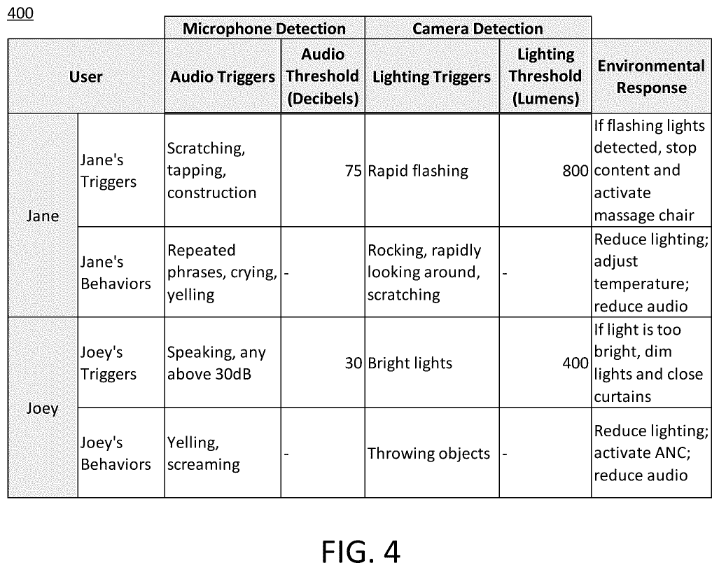

For example, a user sensitive to bright lights may be able to tolerate lights up to 500 lumens, so their lighting threshold may be set to 500 lumens. If ambient environment lighting, content lighting, or some combination thereof exceeds 500 lumens, remedial actions may be deployed in order to prevent or alleviate agitation of the user, for example, such as modifying environment lighting, modifying content brightness, and/or other actions. To determine whether the lighting threshold has been met, environment lighting and content lighting may be compared. Trigger thresholds may be stored in a database (e.g., such as the trigger server 122 and/or such as described with respect to FIG. 4 herein).

For example, the user 301 may be watching a content item on the display 302 (e.g., in a living room in a dwelling). The computer 307 may be responsible for the monitoring of triggers in the content item and the environment and/or for sending commands for remedial actions. The user may be an individual with ASD who uses the headphones 305 to control stimuli in their environment. For example, the user may be watching the content item on the display 302 and listening to the accompanying audio via the speakers 303a . . . 303N. The user 301 may wear the headphones 306 to dampen the noise from the content item's audio and/or to dampen environmental noise.

A triggering event may occur in the content item, for example, sounds greater than a certain audio threshold which may agitate the user. The microphone 304A may detect the audio above the threshold. The camera 304B may detect body movements of the user indicating an increase in agitation which may be associated with the loud content audio. Some individuals with ASD experience repetitive body movements and/or sounds during periods of increased agitation (e.g., meltdowns), which may be detected by the environmental sensors 304A-304B (and other sensors, such as motion sensors, etc.). Based on the detected triggers in the content and/or the detected indicators of increased agitation, the computer 307 may send a command to enable active noise canceling (ANC) on the headphones 306.

The computer 307 may, for example, detect a lighting trigger in the upcoming portion of the content item. The computer 307 may send the command 308 to dim the lights 305a . . . 305N based on the upcoming scene in the content item, for example, an upcoming scene showing gun violence and bright muzzle flashes which would exceed the user's lighting threshold. The computer may send commands to various environmental devices separately or simultaneously, to deploy remedial actions. Although FIG. 3 shows speakers, lights, and headphones, environmental devices may further comprise massage chairs, wearable devices, and/or other devices.

Based on deploying the remedial actions, the computer 307 may continue monitoring the user 301 for indications of decreased agitation. The computer 307 may monitor user reactions to remediation to determine whether to continue deploying remedial actions. Users may submit feedback indicating their preferences regarding remediation. Users may update their triggers, for example, by indicating new triggers and/or new threshold values.

Multiple users 301 may be in the environment 300. For example, the computer 307 may monitor and remediate the triggers of the multiple users 301 simultaneously. A group of users may be watching a content item on the display 302 together. One or more of the users 301 may be wearing headphones 306. One or more of the users 301 may have at least one trigger, and they may experience increases in agitation levels due to their triggers. The computer 307 may determine which users are in the room, what their triggers are, whether they are exhibiting behaviors indicative of agitation, and/or deploy remedial actions based on these factors. For example, if one user is triggered by lights above a certain lighting threshold and another user is triggered by sounds above a certain audio threshold, the computer 307 may determine that an upcoming portion in content contains sounds and lighting above the thresholds. Based on this determination, the computer 307 may deploy remedial actions in an attempt to alleviate the agitation of both users.

Although examples of automatically detected and remediated triggers and user behaviors are discussed herein, users may select to enable remedial actions and/or notify the computer 307 of a meltdown, for example, via a button on a remote control or via a smartphone application. Actions implemented by the computer 307 may be implemented by any other devices described herein, for example, the trigger server 122, the gateway 111, the display 302, and/or other devices.

FIG. 4 is an example table 400 showing detected triggers and remedial actions. The data of the table 400 may be used in determining whether triggers are present in content being presented to a user, in the user's environment, and/or both. Also or alternatively, the table 400 may be used in determining which remedial actions (e.g., environmental responses) to deploy for users depending on detected triggers and/or user behaviors. An environmental response such as dimming the lights may be deployed, for example, if lighting triggers are detected in content and/or in the environment and determined to exceed a lighting threshold. Similarly, an environmental response such as activating active noise cancelling (ANC) on ANC-enabled devices may be deployed, for example, if audio triggers are detected in content in the environment, and/or in a combination of both and determined to exceed an audio threshold. Determining whether triggers exceed a predetermined threshold may comprise detecting triggers in content being output to a user, detecting trigger differences and/or triggers in the user's environment, comparing the detected trigger differences and/or trigger levels with a trigger database (e.g., such as the table 400), and/or deploying remedial actions (e.g., environmental responses) to prevent and/or alleviate user agitation. Specific triggers, trigger types, and/or trigger thresholds for users may be stored in a database such as the table 400. Also or alternatively, users' historical behavioral patterns (e.g., behaviors indicating a user may be agitated) may be stored in the database. The table 400 may be stored in the trigger server 122.

The table 400 shows example users Jane and Joey and their respective triggers and historical behavioral patterns. Triggers may comprise events that cause an increase in agitation in the user (e.g., they may trigger meltdowns). Different types of triggers may include audio triggers, lighting/visual triggers, psychological triggers, and/or other types of triggers. Historical behavioral patterns may include user behaviors that indicate the occurrence or the onset of a period of increased agitation (e.g., a meltdown).

Jane, for example, may be triggered by sounds of scratching, tapping, and construction above a threshold of 75 decibels. She may also be triggered by rapid flashing above 800 lumens. Environmental responses may be implemented, for example, if Jane's triggers are detected in content she is watching and/or in her environment (e.g., by the computer 307).

If, for example, a display is outputting content at 40 dB but the ambient audio level is determined to be below 10 dB, Jane may be triggered by the difference in audio levels. Remedial actions may be deployed, for example, to lower the content audio to match the ambient audio and/or to increase ambient audio (e.g., enabling environmental audio devices such as noise makers, etc.) to match the content audio.

For example, if she is watching content at a level of 40 dB, and ambient noise is at 10 dB, the computer 307 may determine that no environmental responses are necessary. If, for example, construction equipment starts being used nearby, sensors such as the microphone 304A may detect the occurrence and volume level of the environmental audio. The computer 307 may determine that the environmental audio or the combined audio from the content and Jane's environment satisfy her audio threshold, for example, based on comparing the detected audio levels with the table 400. Remedial actions may be deployed based on the detected triggers, for example, such as specific actions known to soothe Jane (e.g., enabling a massage chair), modifying or stopping the content audio, and/or other actions. A similar process may be employed to deploy remedial actions based on determining whether visual triggers exceed a visual trigger threshold for a user.

Environmental responses may comprise changes to the environment and/or to the content in order to alleviate and/or prevent a user's agitation. For example, environmental responses for Jane may include stopping the content she is watching and activating a massage chair if flashing lights above the threshold are detected (e.g., in the content and/or in her environment). Similarly, when Jane is agitated, Jane's behaviors indicating agitation may include repeating phrases, crying, and yelling. Predetermined environmental responses may be implemented, for example, if these behaviors are detected (e.g., by the computer 307). Lighting may be reduced, temperature may be adjusted, and/or content audio may be reduced, for example, if Jane appears to be entering a period of increased agitation. As described herein, environmental responses may be implemented for multiple users separately or simultaneously based on their triggers and/or historical behavior patterns. If Jane is exhibiting behaviors indicating that she is already in an agitated state, then the environmental responses may be adjusted accordingly, and may occur more frequently, earlier, with greater intensity, etc.

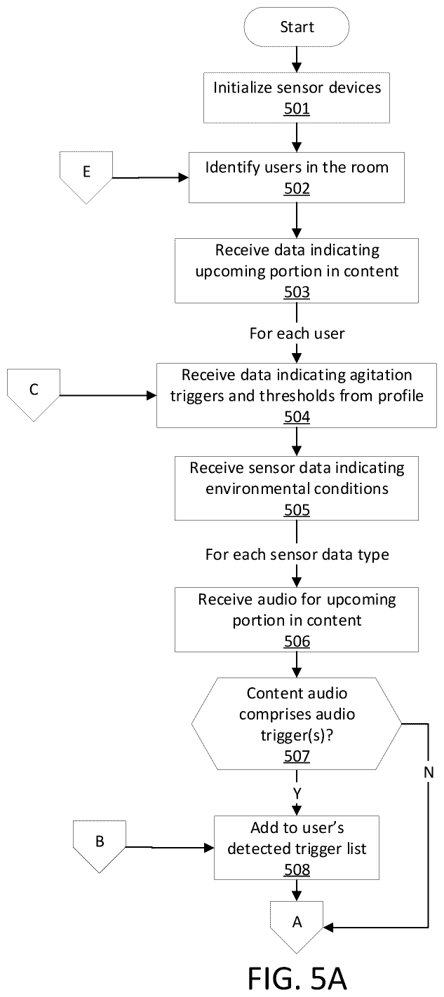

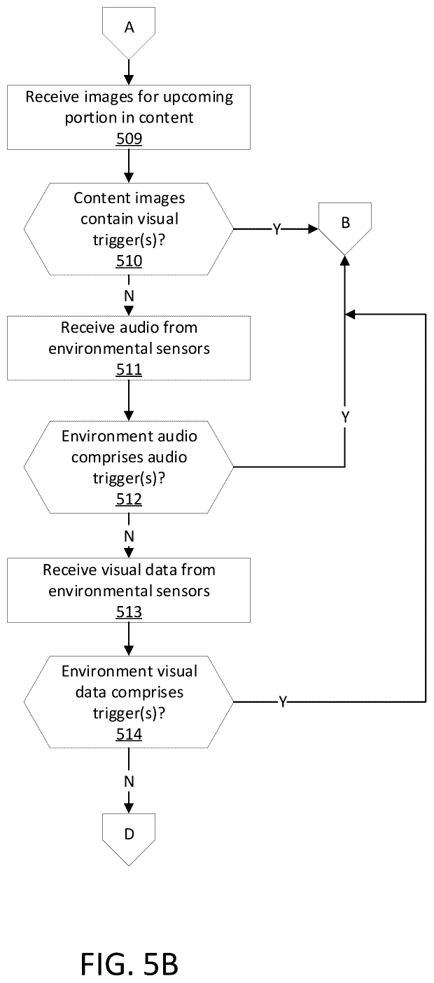

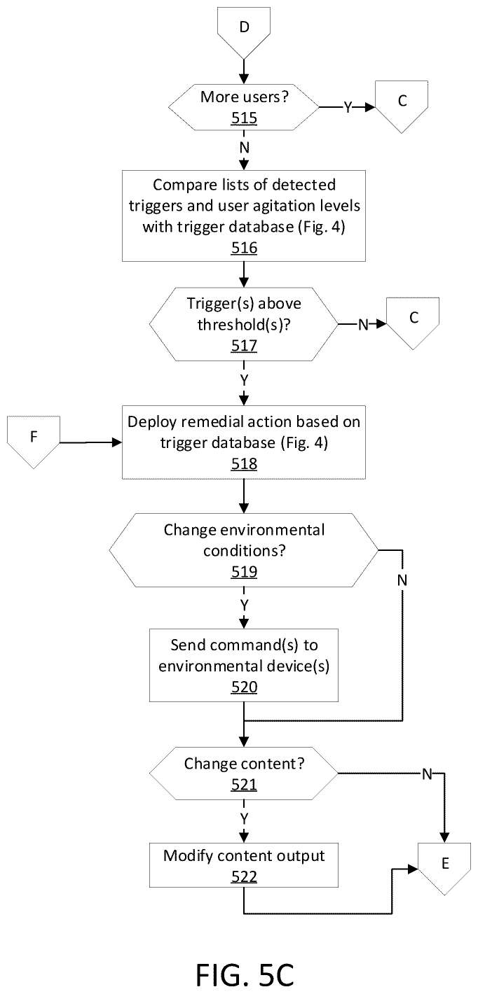

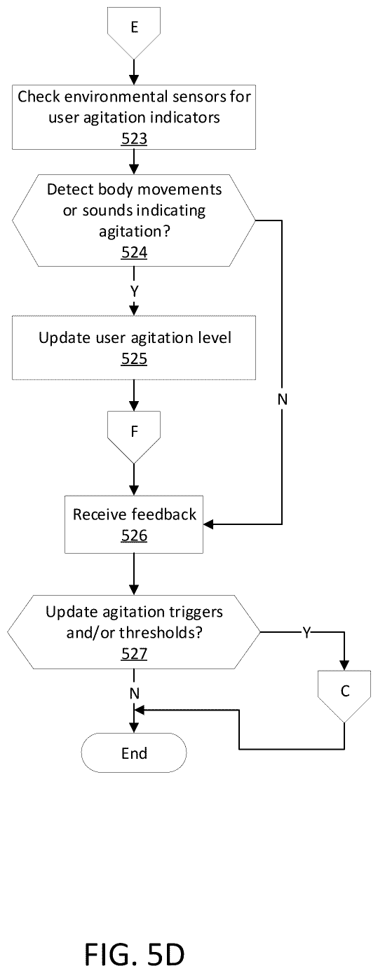

FIGS. 5A-5D are flow charts showing an example method for remediation based on detected triggers. Triggers may be detected in content being output to users and/or in the users' environment. Triggers and detected in content and their corresponding trigger levels may be compared with triggers detected in the environment and their corresponding trigger levels to determine whether either of the trigger levels or the combined trigger levels meet a user's predetermined threshold. For example, content audio trigger levels may be compared with ambient audio trigger levels to determine if an audio threshold has been met by the content audio trigger levels, the ambient audio trigger levels, and/or a combination of the content and ambient audio trigger levels. Similarly, content visual trigger levels may be compared with ambient visual trigger levels to determine if a visual trigger threshold has been met. Remediation may be implemented in order to prevent and/or alleviate agitation in the users. The method of FIGS. 5A-5D may be implemented by the computer 307 and/or any other devices described herein.

At 501, sensor devices may be initialized. For example, the microphone 304A and/or the camera 304B may be initialized (e.g., by the computer 307 and/or any other devices described herein). Sensor devices may include microphones, cameras, motion sensors, wearable devices, and/or other devices. Initialization may comprise loading the table 400, determining environmental control (e.g., which devices in the environment may be controlled during an environmental response), bringing sensor devices online (e.g., the microphone 304A, the camera 304B, etc.), bringing environmental devices online (e.g., the speakers 303a-N, the lights 305a-N, the headphones 306, etc.), detecting a user's initial agitation state, detecting the environment's initial triggers, and/or other initialization activities. At 502, users in the room may be identified. For example, the sensors may identify users based on their visual appearance, voices, user devices, and/or other factors. At 503, data indicating upcoming portions in content may be received. For example, advance monitoring of the upcoming portions may occur. Advance monitoring may include, for example, analyzing portions a predetermined time in advance (e.g., the next five minutes of upcoming content). For example, advance monitoring may allow for environmental responses that take time to implement (e.g., modifying the temperature via an air conditioning system). The data may indicate audio and/or audio levels, lighting patterns and/or lighting levels, triggering events (e.g., violence, etc.), and/or other factors. The data may comprise manifest files for the upcoming portions in the content. The manifest files may indicate potential triggers in the upcoming portion in the content, such as whether a portion of a movie contains blood, violence, certain types of sound, color patterns, flashing lights, etc. For each identified user, at 504, data may be received indicating their agitation triggers and thresholds (e.g., from a profile). Data from the table 400, for example, may indicate agitation triggers and thresholds for identified users.

At 505, sensor data indicating environmental conditions may be received. For example, environmental conditions may include content being output to the user, ambient lighting, ambient sounds, and/or other conditions. Ambient lighting, for example, may be detected by light sensors. The content being output to the user may be detected, for example, based on performing image recognition on the video stream. For each data type detected by sensors, a loop may be implemented. At 506, audio for the upcoming portion in the content may be received. At 507, a determination may be made as to whether the audio for the upcoming portion comprises one or more audio triggers. For example, audio triggers may be detected, for example, by comparing codes in a manifest file for a content item with codes in a user profile. Also or alternatively, content audio may be processed, for example, by the computer 307, to detect the presence of a triggering sound. In the case of a “Yes” determination, at 508, the detected triggers may be added to a list of detected triggers for that user and the algorithm may proceed to 509. For example, a session for keeping the list may begin when a user starts watching a content item, and during the session, the list may be appended as various triggers occur in the content. Also or alternatively, the list may keep track of the user's agitation state as related to the various triggers encountered in the content. Also or alternatively, the list may keep track of total trigger levels, for example, such as total audio trigger levels from the environment and content and/or total visual/lighting trigger levels. Monitoring the total trigger levels, for example, in addition to individual trigger levels, may allow for environmental responses to be deployed if the total level exceeds a user's threshold as shown in the table 400 (e.g., even if individual trigger levels are below thresholds). For example, the list may be based on the table 400 or it may be a portion of the table 400. In the case of a “No” determination, at 509, images for the upcoming portion in the content may be received.

At 510, a determination may be made as to whether the images for the upcoming portion comprise one or more visual and/or lighting triggers. For example, image recognition may be performed on the video stream to determine whether certain triggers, lighting patterns, brightness levels, and or other factors occur in the content item. Also or alternatively, visual and/or lighting triggers may be identified based on codes in the manifest files for the content item. In the case of a “Yes” determination, the detected triggers may be added to the user's list of detected triggers at 508 and the algorithm may proceed to 511. In the case of a “No” determination, at 511, audio from the environmental sensors may be received. For example, the microphone 304A may retrieve audio from the room in which the user is watching the content (e.g., ambient audio).

At 512, a determination may be made as to whether the environmental audio comprises audio triggers (e.g., construction noises for Jane, etc.). For example, the sound recognition may be performed on the environmental audio to detect sound patterns associated with various triggers. Also or alternatively, the environmental audio may be compared with saved audio files to determine whether certain triggers occur. In the case of a “Yes” determination, the detected triggers may be added to the user's list of detected triggers at 508 and the algorithm may proceed to 513. In the case of a “No” determination, at 513, visual data from the environmental sensors may be received. For example, the camera 304B may retrieve video from the room in which the user is watching the content (e.g., ambient conditions).

At 514, a determination may be made as to whether the environmental video comprises visual and/or lighting triggers, as described in the table 400. For example, someone may have turned on the room light to above 400 lumens, which is above Joey's lighting threshold. A lighting sensor, for example, may determine whether environmental lighting exceeds a predetermined threshold. Also or alternatively, image recognition may be performed on the environmental video in order to detect any triggers (e.g., a moving vacuum cleaner in the room). In the case of a “Yes” determination, the detected triggers may be added to the user's list of detected triggers at 508 and the algorithm may proceed to 515. In the case of a “No” determination, at 515, a determination may be made as to whether there are more identified users. In the case of a “Yes” determination, the loop beginning at 504 may repeat for the additional users. In the case of a “No” determination, at 516, the lists of detected triggers may be compared with a trigger database. For example, the trigger server 122 may comprise a trigger database such as the table 400. The lists of detected triggers may comprise lists of content triggers and their corresponding trigger levels, lists of ambient/environmental triggers and their corresponding trigger levels, and/or lists of the combined content and ambient triggers and trigger levels. At 516, the lists may be compared to a trigger database such as the table 400 to determine whether a user's trigger threshold has been met by either the content triggers, the ambient environmental triggers, and/or the combination of content and ambient environmental triggers.

Also or alternatively, any of the triggers and/or types of triggers described herein may be flagged by a user (e.g., using a remote control, an app, etc.) to identify when a trigger occurs, for example, the user may press a “trigger” button to indicate when a triggering sounds (e.g., a dog barking) has occurred in the environment and/or in the content. Also or alternatively, the trigger recognition described herein may be performed by the computer 307, a gateway, a smartphone, and/or other devices described herein.

At 517, a determination may be made as to whether one or more of the detected triggers are above the thresholds. The thresholds may indicate a maximum trigger level that a user can tolerate. For a user with an audio trigger threshold of 75 dB, for example, at 517, the determination may comprise comparing the content audio and the environmental audio to determine whether the content audio level, the environmental audio level, and/or the total audio level exceeds 75 dB. Similarly, for a user with a lighting trigger threshold of 800 lumens, for example, at 517, the determination may comprise comparing the content lighting and the environmental lighting to determine whether either the content lighting level, the environmental lighting level, and/or the total lighting level exceeds 800 lumens. The comparison and determination may be performed for any type of trigger and any trigger level.

In the case of a “Yes” determination, at 518, remedial actions may be deployed, for example, based on the environmental responses of the table 400. In the case of a “No” determination, the loop beginning at 504 may repeat until one or more triggers are detected. At 519, a determination is made as to whether to alter environmental conditions. Altering environmental conditions may include, for example, adjusting ambient lighting, temperature, active noise cancellation, massage furniture, wearable devices, and/or other actions. In the case of a “Yes” determination, at 520, one or more commands may be sent to environmental devices to alter environmental conditions. For example, the command 308 may be sent to the lights 305a . . . 305N to enable dimming based on detected triggers and user behaviors.

At 521, a determination may be made as to whether to modify the content being output to the user. For example, the list of detected triggers may be analyzed to determine whether any warrant a remedial action (e.g., an environmental response) as described for the table 400. Modifying the content may include, for example, adjusting the audio level, adjusting brightness, adjusting contrast, skipping past triggering scenes, replacing triggering content with alternate content such as calming content (e.g., white noise, water sounds, etc.), and/or other actions. In the case of a “Yes” determination, at 522, the content may be modified and the algorithm may proceed to 523. For example, the content audio may be lowered to below Jane's threshold such that it is no longer at or above the triggering level. In the case of a “No” determination, at 523, data from environmental sensors may be checked for indications of user agitation.

At 524, a determination may be made as to whether environmental sensors indicate user behavioral patterns (e.g., body movements and/or sounds) indicating user agitation. For example, data from the sensors 3034A-304B indicating sounds relating to Jane may be searched for crying, yelling, and/or repeating phrases in order to determine the occurrence and/or onset of a period of increased agitation. Also or alternatively, the sensor data may be separated based on user, for example, if both Joey and Jane are in the room at the same time. The data may be analyzed using image recognition, sound recognition, codes/tags, and/or other methods. In the case of a “Yes” determination, at 525, the user's agitation level may be updated and the algorithm may proceed to 526. Based on the updated agitation levels for each identified user, at 518, remedial actions may be deployed. In the case of a “No” determination, at 526, feedback may be received. Feedback may comprise, for example, indications that certain triggers may have been missed detection. Also or alternatively, feedback may be received via user input at a remote control, an app, a survey, and/or other methods. For example, a user may use an app to indicate that a triggering lighting pattern in a content item may have missed detection. The feedback may be analyzed, and the missed trigger may be tagged and added to the user's trigger list and/or the table 400 for future reference. Also or alternatively, a user's agitation level may be detected separate from a trigger (e.g., Jane may scream even if no triggers are detected in the environment or the content). The system may then review the past portions of content and/or environmental data to determine whether a trigger occurred, for example, at a threshold lower than the predetermined threshold. IN this case, the system may prompt the user to confirm whether a trigger has missed detection in order to update the trigger database and/or thresholds. At 527, a determination is made as to whether to update the user's agitation triggers and/or thresholds. The agitation triggers and/or thresholds may be comprised in the table 400, for example, and/or implemented by the trigger server 122. In the case of a “Yes” determination, the updated trigger profile (e.g., the table 400) may be received at 504, and the remediation may proceed based on the updated trigger profile.

Although examples are described above, features and/or steps of those examples may be combined, divided, omitted, rearranged, revised, and/or augmented in any desired manner. Various alterations, modifications, and improvements will readily occur to those skilled in the art. Such alterations, modifications, and improvements are intended to be part of this description, though not expressly stated herein, and are intended to be within the spirit and scope of the disclosure. Accordingly, the foregoing description is by way of example only, and is not limiting.

What is claimed is:

1. A method comprising:
receiving, by a computing device, information indicating an upcoming portion of content being output to a user in an environment;
determining that the upcoming portion of content is associated with one or more trigger conditions associated with the user;
comparing the one or more trigger conditions associated with the upcoming portion of content with one or more ambient conditions associated with the environment;
determining, based on the comparing, that a trigger threshold is satisfied; and
sending, based on the thresholding being satisfied, a command to cause a modification in the content or the environmental device.

2. The method of claim 1, wherein the sending is further based on:
receiving a video image of the upcoming portion of content; and
detecting, in the video image, a visual trigger associated with the user.

3. The method of claim 1, wherein the sending is further based on:
receiving an audio file of the upcoming portion of content; and
detecting, in the audio file, an audio trigger associated with the user.

4. The method of claim 1, wherein the comparing further comprises:
comparing audio of the upcoming portion of content with ambient audio of the environment; and
comparing visual conditions of the upcoming portion of content with ambient visual conditions of the environment.

5. The method of claim 1, wherein the determining further comprises:
determining whether audio of the upcoming portion of content or ambient audio of the environment or a combination thereof satisfies an audio threshold;
determining whether a difference between the audio conditions of the upcoming portion of content and ambient audio conditions of the environment satisfies the audio threshold;
determining whether visual conditions of the upcoming portion of content or ambient visual conditions of the environment or a combination thereof satisfies a visual threshold; and
determining whether a difference between the visual conditions of the upcoming portion of content and ambient visual conditions of the environment satisfies the visual threshold.

6. The method of claim 1, wherein the sending on the command comprises:
sending a command to adjust audio of the upcoming portion of content.

7. The method of claim 1, wherein sending the command comprises causing an audio device to output calming audio.

8. The method of claim 1, wherein the sending the command comprises:
sending a command to adjust a noise cancellation setting of an audio headphone of the user.

9. A method comprising:
receiving, by a computing device, a list of trigger conditions of a user indicating maximum audiovisual thresholds;
receiving information indicating an upcoming portion of content being output to the user;
receiving information indicating ambient conditions of the user's environment;
comparing the upcoming portion of content and the ambient conditions with the list of trigger conditions associated with the user; and
changing, based on the comparing, the output of the content.

10. The method of claim 9, wherein the receiving information indicating the upcoming portion of content further comprises:
receiving audio data associated with the upcoming portion; and
receiving video data associated with the upcoming portion.

11. The method of claim 9, wherein the receiving information indicating the upcoming portion of content further comprises receiving metadata indicating triggers occurring in the upcoming portion of content.

12. The method of claim 9, wherein the receiving information indicating the ambient conditions of the environment further comprises:
receiving audio data from one or more audio sensors; and
receiving visual data from one or more visual sensors.

13. The method of claim 9, wherein the comparing further comprises:
comparing audio of the upcoming portion of content with ambient audio of the environment;
comparing visual conditions of the upcoming portion of content with ambient visual conditions of the environment; and
determining that differences between the ambient conditions and the upcoming portion of content satisfy predetermined thresholds.

14. The method of claim 9, further comprising sending a command to an environmental audio device to output calming audio.

15. A method comprising:
receiving, by a computing device, sensor information ambient conditions of a user's environment;
activating, based on the ambient conditions, advance monitoring of upcoming scenes in content being output to the user; and
based on the advance monitoring:
sending a command to control an environmental device to adjust an environment in which the user will receive an upcoming scene; and
changing output of the content.

16. The method of claim 15, wherein the receiving is further based on:
receiving a video image of the user's environment;
detecting, in the video image, a visual trigger of the user; and
determining that the visual trigger satisfies a visual trigger threshold of the user.

17. The method of claim 15, wherein the receiving is further based on:
receiving an audio recording of the user's environment;
detecting, in the audio recording, an audio pattern of an audio trigger of the user; and
determining that the audio trigger satisfies an audio trigger threshold of the user.

18. The method of claim 15, wherein the sending is further based on:
determining an initial agitation state of the user; and
determining that the upcoming scene would increase the agitation state beyond an agitation threshold of the user.

19. The method of claim 15, wherein the sending the command comprises:
sending a command to adjust a noise cancellation setting of an audio headphone of the user.

20. The method of claim 15, wherein sending the command comprises causing an audio device to output calming audio.
